(19) United States
(12) Patent Application Publication
(10) Pub. No.: US 2011/0015485 A1
(43) Pub. Date: Jan. 20, 2011

(54) Endoscopic Device and Image Processing Method Thereof

(75) Inventor: CHIH-CHUN CHAN, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignee: MEDICAL INTUBATION TECHNOLOGY CORPORATION, Taoyuan County (TW)

(21) Appl. No.: 12/837,204

(22) Filed: Jul. 15, 2010

(30) Foreign Application Priority Data
Jul. 15, 2009 (TW) 098124007

(51) Int. Cl. A61B 1/04

(52) U.S. Cl. 600/109

(57) ABSTRACT

An endoscopic device and its image processing method include a casing, a sensing module for reflecting a light from a target object through a reflecting unit, capturing an optical image, and converting the optical image into an electric signal, a switching unit installed on a side of the casing, an image processing unit installed in the casing and electrically coupled to the switching unit, for encoding the electric signal into an image signal and a display unit installed on another side of the casing. If the switching unit is triggered, the switching unit outputs a switch signal, and the image processing unit mirrors the image signal according to the switch signal to obtain a mirrored image signal, and the display unit receives the mirrored image signal and outputs a mirrored image. If the switching unit is not triggered, the display unit receives the image signal and outputs an image.

### BACKGROUND OF THE INVENTION

1. Field of the Invention

The present invention relates to an endoscopic device and an image processing method applied for the endoscopic device, and more particularly to an endoscopic device and an image processing method applied for the endoscopic device capable of processing an original image by a mirror process into an image normally recognizable by users.

2. Description of the Related Art

A conventional endoscopic device is a specially made tube and comprised of a camera device and a light source and provided for displaying an internal structure of a body after a display device is connected, so that a doctor can diagnose a patient's disease from an image shown on a screen. The endoscopic device can be used for examining an examinee's organ, as long as the organ has an opening connected to an exterior of the examinee's body. For example, a digestive tract endoscopic device (used for examining an esophagus, a stomach and a duodenum) is passed through a mouth, and a colonoscopy is passed through an anus. If there is no opening, an opening can be formed by surgical operation. For example, a gastroscopy requires making an opening at the examinee's abdomen, and an appendectomy requires cutting the skin around the examinee's joint. In addition, the endoscope also can be used for industrial applications. Much time and cost will be taken to disassemble instruments when it is necessary to observe the interior of large or precision instruments, but the endoscope simply requires a camera lens and a soft tube to be extended through a gap to observe the interior of the instruments.

Sometimes, it is necessary to extend the lens and the soft tube deep into the gap to view the interior of the instruments from other angles, and a reflective lens is installed at the front of the camera lens and provided for viewing a target object disposed at other angles. However, an image projected onto a screen is a mirrored image having left and right sides opposite to an actual image, such that a wrong recognized direction may be resulted and the operation schedule may be affected when doctors or engineers examine a patient's body or perform an industrial test respectively.

### SUMMARY OF THE INVENTION

Therefore, it is a primary objective of the invention to overcome the aforementioned shortcomings and deficiencies of the conventional endoscope by providing an endoscopic device and an image processing method thereof, so as to overcome the problem of producing a mirrored image when an image is projected onto a screen.

To achieve the foregoing objectives, the present invention provides an endoscopic device, comprising a casing, a sensing module, a switching unit, an image processing unit and a display unit. The sensing module captures an optical image according to a reflecting light reflected from a target object and converts the optical image into an electric signal. The switching unit is installed on a side of the casing, and the image processing unit is installed in the casing and electrically coupled to the switching unit, and the electric signal is encoded to an image signal, and a display unit is installed on another side of the casing. If the switching unit is triggered, the switching unit outputs a switch signal, and the image processing unit mirrors the image signal to obtain a mirrored image signal according to the switch signal, and the display unit receives the mirrored image signal and outputs a mirrored image. If the switching unit is not triggered, then the display unit will receive the image signal and output an image.

The sensing module includes a light source, a camera lens and an optical sensing element, wherein the light source projects lights onto a target object, and the light of the target object reflected by the reflecting unit forms an optical image at the camera lens, and the optical sensing element converts the optical image into an electric signal.

The light source is a light emitting diode (LED) or an organic light emitting diode (OLED).

The optical sensing element is a charge coupled device (CCD) or a complementary metal oxide semiconductor (CMOS) photoelectric diode.

The transmission line transmits the electric signal to the image processing unit.

The endoscopic device further comprises a transmission line for transmitting an electric signal from the sensing module to the image processing unit.

The transmission line is an optical fiber cable or a copper-wire cable.

The mirrored image is an image formed by horizontally turning over the optical image.

The mirrored image is an image formed by vertically turning over the optical image.

The reflecting unit is installed on a side of the sensing module, and the reflecting unit is a reflective lens.

Another objective of the present invention is to provide an image processing method applied for an endoscopic device, and the method comprises the following steps. Firstly, a light is reflected from a target object to a sensing module by a reflecting unit, and the sensing module captures an optical image and converts the optical image from a sensing module into an electric signal, and the electric signal is transmitted to an image processing unit. Secondly, the image processing unit encodes the electric signal to obtain an image signal. Thirdly, the switching unit is triggered, and the image processing unit obtains a mirrored image signal according to the mirrored image signal. Finally, a display unit is provided for receiving the mirrored image signal, and outputting a mirrored image.

The image processing method applied for an endoscopic device image further comprises the following steps. Firstly, the switching unit is turned off. Finally, the display unit receives the image signal and outputs an image.

The electric signal is transmitted from the sensing module to the image processing unit through a transmission line.

The endoscopic device having a mirror displaying image in accordance with the present invention has one or more of he following advantages:

(1) The endoscopic device performs a mirroring processing to an image passing through the reflective lens into an image normally recognizable by users to improve the recognition of a displayed image.

(2) The endoscopic device adopts the switching unit to allow users to freely select the use of mirroring processing.

### BRIEF DESCRIPTION OF THE DRAWINGS

FIG. 1 is a schematic view of an endoscopic device in accordance with a first preferred embodiment of the present invention;

FIG. 2 is a block diagram of an endoscopic device in accordance with a first preferred embodiment of the present invention;

FIG. 3 is a schematic view of a target object, an optical image, a mirrored image and an image in accordance with a first preferred embodiment of the present invention;

FIG. 4 is a flow chart of an image processing method applied for an endoscopic device in accordance with a first preferred embodiment of the present invention;

FIG. 5 is a schematic view of an endoscopic device in accordance with a second preferred embodiment of the present invention;

FIG. 6 is a block diagram of an endoscopic device in accordance with a second preferred embodiment of the present invention; and

FIG. 7 is a flow chart of an image processing method applied for an endoscopic device in accordance with a second preferred embodiment of the present invention.

### DETAILED DESCRIPTION OF THE PREFERRED EMBODIMENTS

The technical characteristics of the present invention will become apparent with the detailed description of preferred embodiments and the illustration of related drawings as follows.

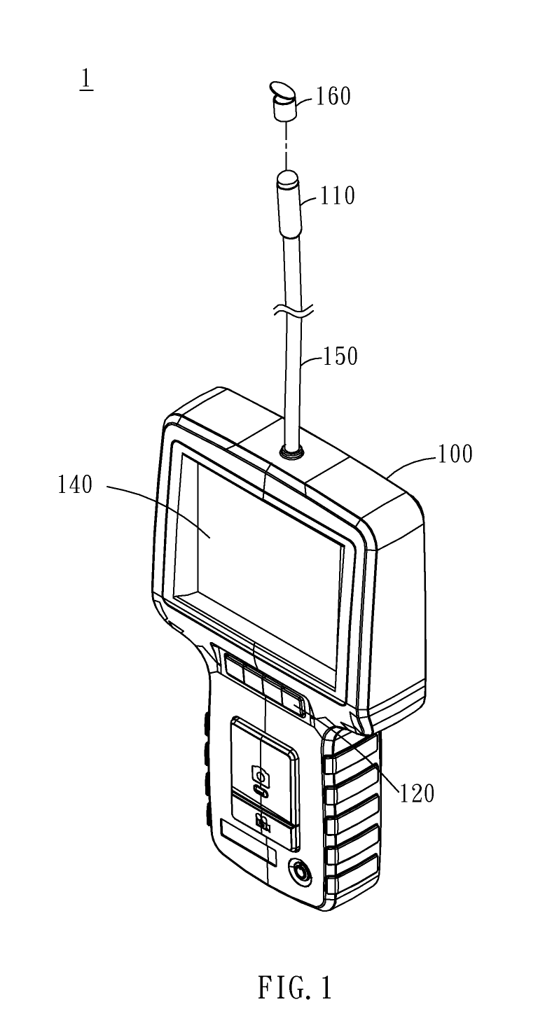

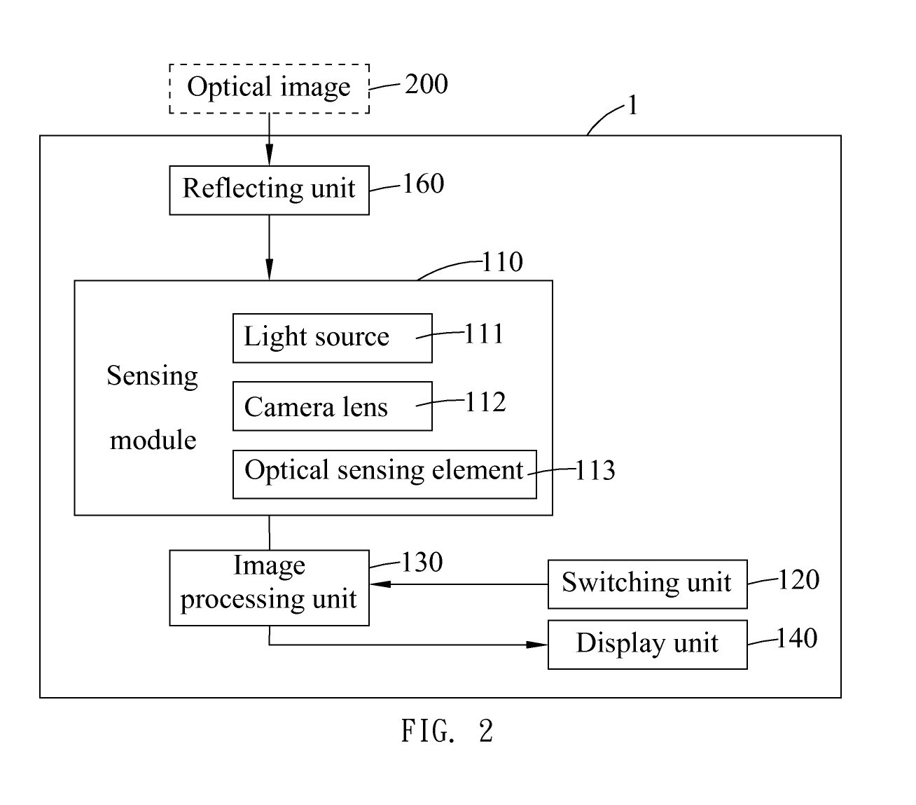

With reference to FIGS. 1 and 2 for a schematic view and a block diagram of an endoscopic device in accordance with a first preferred embodiment of the present invention respectively, the endoscopic device 1 comprises a casing 100, a sensing module 110, a switching unit 120, an image processing unit 130 and a display unit 140. The sensing module 110 and the casing 100 are coupled by a transmission line 150, and the switching unit 120 is installed on a side of the casing 100, and the image processing unit 130 is installed in the casing 100 and electrically coupled to the switching unit 120, and the display unit 140 is installed on another side of the casing 100. The endoscopic device 1 further comprises a reflecting unit 160 installed on a side of the sensing module 110, such that the reflecting unit 160 can be installed for the application of the endoscopic device 1 to observe a target object disposed at other angles.

The sensing module 110 comprises a light source 111, a camera lens 112 and an optical sensing element 113. In the embodiment having the installed reflecting unit 160, the light source 111 projects lights onto a target object 2, such that after the light of the target object 2 reflected from the reflecting unit 160 is received by the camera lens 112, an optical image 200 is formed, and then the optical sensing element 113 converts the optical image 200 into an electric signal, wherein the light source 111 is a light emitting diode (LED) or an organic light emitting diode (OLED), and the optical sensing element 113 is a charge coupled device (CCD) or CMOS photoelectric diode. However, the present invention is limited to such arrangement only, but the light source 111 or the optical sensing element 113 can be changed according to the manufacturing and practical requirements. The switching unit 120 is installed on a side of the casing 100 and provided for switching a display mode. The image processing unit 130 is installed in the casing 100 and electrically coupled to the switching unit 120, for encoding the electric signal into an image signal. The display unit 140 is installed on a side of the casing 100 and provided for receiving an image signal and outputting an image for a user's viewing. The transmission line 150 is provided for transmitting the electric signal from the sensing module 110 to the image processing unit 130, wherein the transmission line 150 can be an optical fiber cable or a copper-wire cable, or a cable made of other materials depending on the practical application requirements. In addition, the reflecting unit 160 can be a reflective lens.

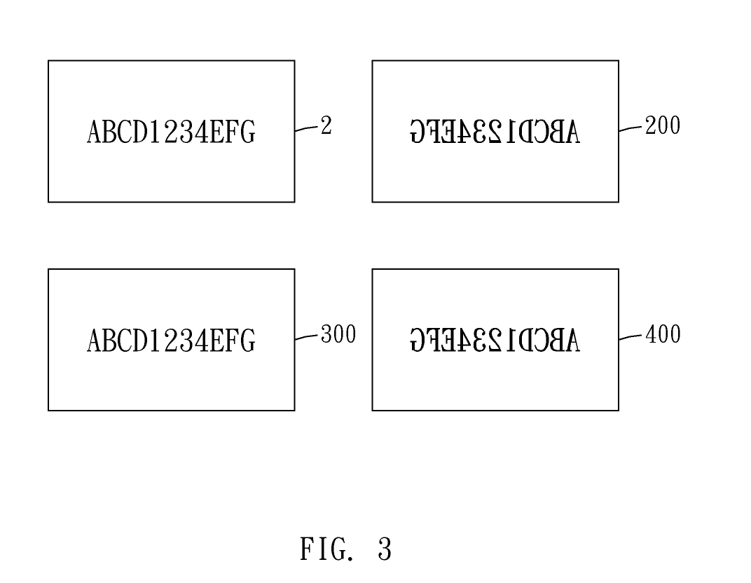

After the electric signal enters into the image processing unit 130, and the switching unit 120 is triggered, the switching unit 120 outputs a switch signal, and the image processing unit 130 mirrors the image signal according to the switch signal to obtain a mirrored image signal, and the display unit 140 receives a mirrored image signal and outputs a mirrored image 300. If the switching unit 120 is not triggered, the display unit 140 receives an image signal and outputs an image 400, wherein the image 400 will not be mirrored, but the image 400 will be outputted in the same direction of the optical image 200. With reference to FIG. 3 for a schematic view of a target object, an optical image, a mirrored image and an image in accordance with a preferred embodiment of the present invention, the target object 2 can be a serial number of a component, and the serial number is disposed on a lateral side of the component, so that a user requires installing a reflecting unit 160 to the endoscopic device 1. With the installation of the reflecting unit 160, an optical image 200 reflected to the sensing module 110 is an image formed by horizontally turning the target object 2 by 180 degrees. If the switching unit 120 is not triggered, the display unit 140 simply displays an image 400 formed by horizontally turning over the target object 2 by 180 degrees, but the image 400 has a relation of turning over the target object 2 by 180 degrees, so that it is difficult to recognize the image 400 directly. After the switching unit 120 is triggered, the display unit 140 can display a mirrored image 300 mirrored by the image processing unit 530. Now, the mirrored image 400 is like the target object 2, so that users can recognize it directly. This preferred embodiment only illustrates that the mirrored image 300 is an image formed by horizontally turning over the optical image 200, but the mirrored image 300 can be an image formed by vertically turning over the optical image 200 in accordance with another preferred embodiment not shown in the figures. In addition, the image processing unit 130 can use another switch signal to perform a digital zoom to the image processing signal and outputs the image processing signal to a screen, in order to amplify the image for the user's viewing in the screen. The switching unit 120 can be designed with ON/OFF, and the mirror mode and zoom mode can be changed according to the time of pressing the switching unit 120. The present invention is not limited to such arrangements only, but any other equivalent switching method can be used for triggering the mirror mode or the zoom mode.

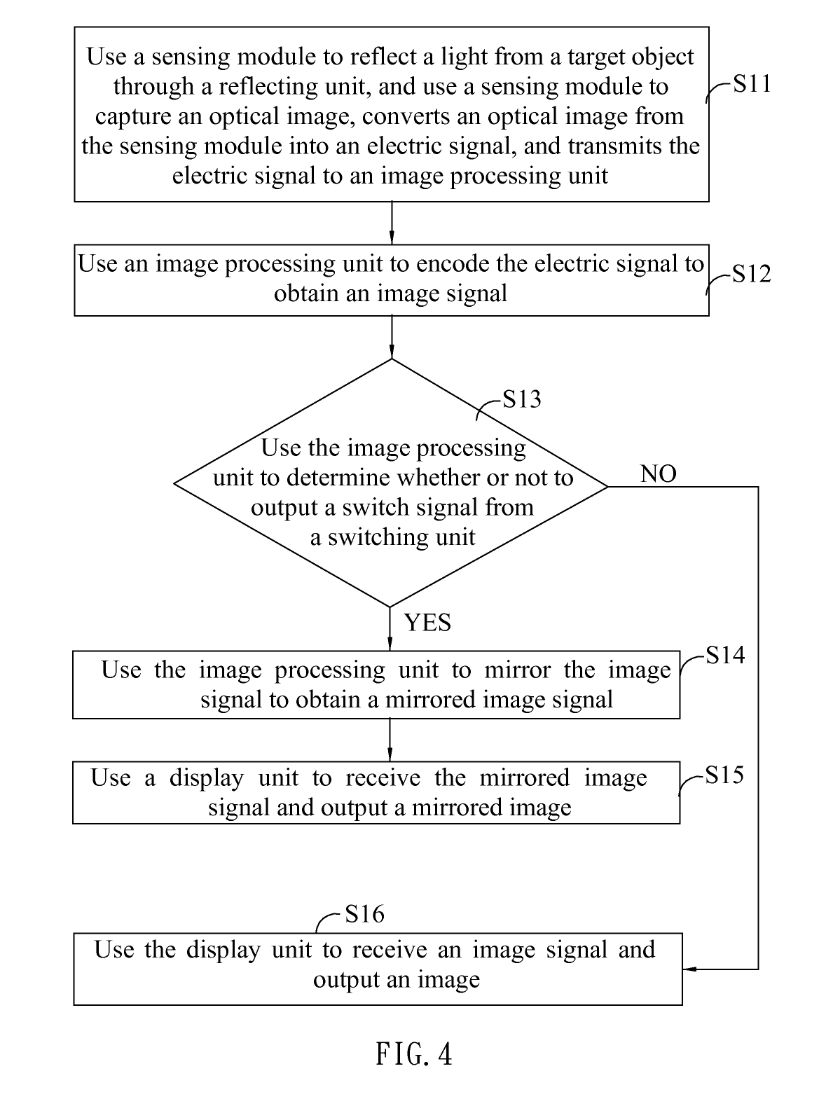

With reference to FIG. 4 for a flow chart of an image processing method applied for an endoscopic device in accordance with a first preferred embodiment of the present invention, the image processing method applied for the endoscopic device image comprises the following steps:

S11: Use a sensing module to reflect a light from a target object through a reflecting unit, and use a sensing module to capture an optical image, convert the optical image through the sensing module into an electric signal, and transmit the electric signal to an image processing unit.

S12: Use an image processing unit to encode the electric signal to obtain an image signal.

S13: Use the image processing unit to determine whether or not to output a switch signal from a switching unit.

S14: Use the image processing unit to mirror the image signal to obtain a mirrored image signal.

S15: Use a display unit to receive the mirrored image signal and output a mirrored image.

In addition, the image processing method applied for an endoscopic device executes the following steps, if the image processing unit has not received the switch signal.

S16: Use the display unit to receive an image signal and output an image.

In this image processing method applied for an endoscopic device image, the following method is used to achieve the effect of displaying the image in the same direction as the target object. If the light source projects lights onto the target object, a light will be reflected to the camera lens through a reflecting unit to form an optical image. The optical image is sensed by the optical sensing element and converted into an electric signal, and the electric signal is transmitted to the image processing unit through a transmission line, and the image processing unit encodes the electric signal to obtain an image signal. If the switching unit is triggered, the image processing unit will mirror the image signal to obtain a mirrored image signal. The mirrored image signal is received by the display unit and a mirrored image is outputted to the display unit for a user's viewing. If the switching unit is not triggered, the image signal will not be mirrored, but the display unit will receive the image signal and output an image.

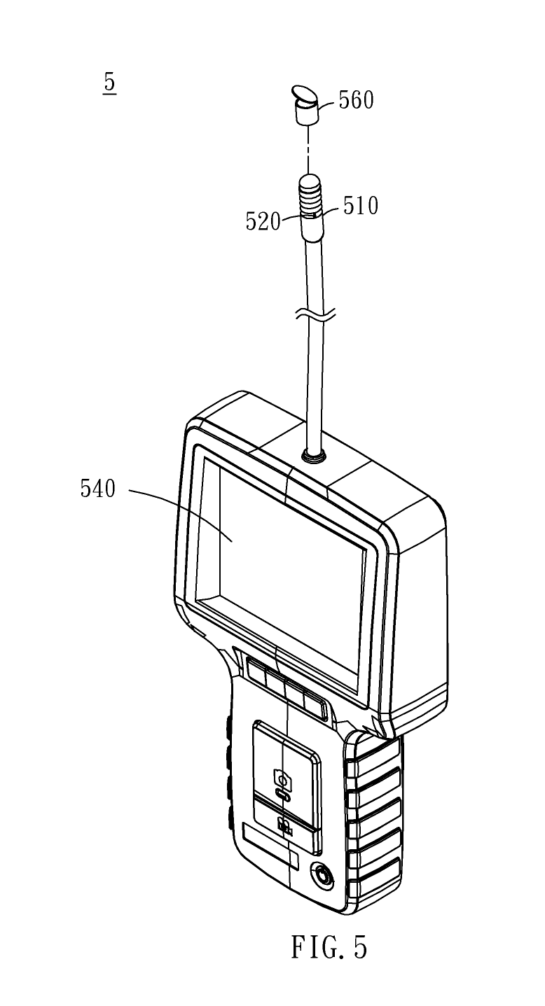

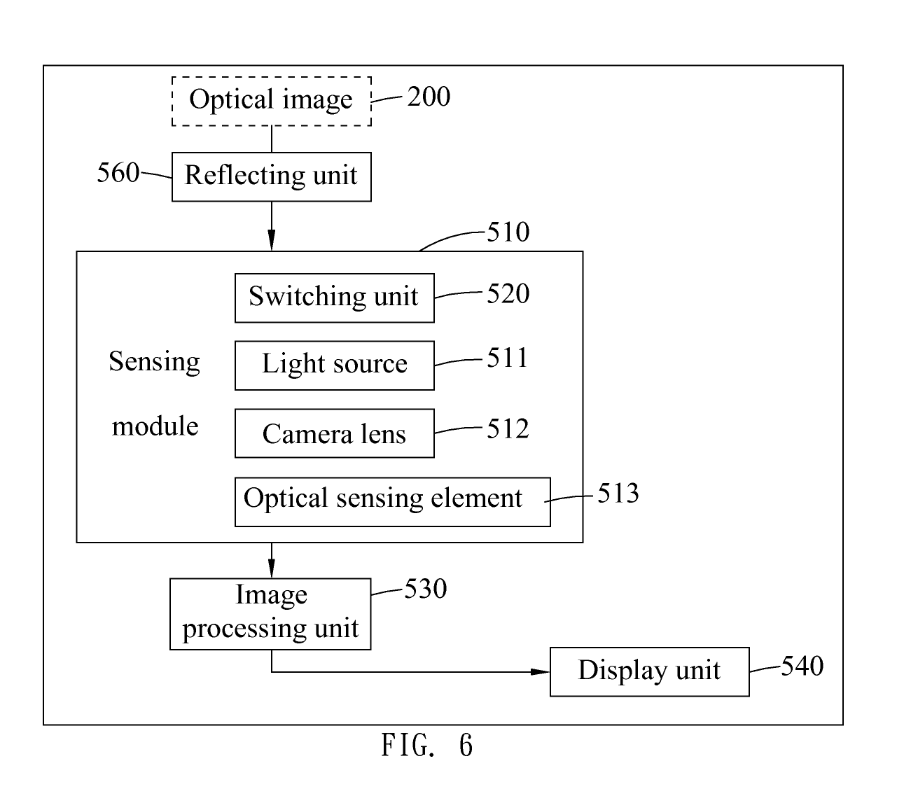

With reference to FIGS. 5 and 6 for a schematic view and a block diagram of an endoscopic device in accordance with a second preferred embodiment of the present invention respectively, a switching unit 520 is installed on a side of a sensing module 510, such that when a reflecting unit 560 is latched or screwed to the sensing module 510, the latching or screwing direction will trigger the switching unit 520, so that after an electric signal of an optical image 200 enters into an image processing unit 530, the image processing unit 530 mirrors an image signal according to a switch signal to obtain a mirrored image signal, and then a display unit 540 receives a mirrored image signal and outputs a mirrored image. If the reflecting unit 560 is not latched to the sensing module 510, the switching unit 520 will not be triggered, so that the display unit 540 receives the image signal and outputs an image 400, and the image 400 will not be mirrored, but the image 400 will be outputted in the same direction of the optical image 200. The sensing module 510 also includes a light source 511, a camera lens 512 and an optical sensing element 513. The installation and operation of the remaining components are the same as those of the first preferred embodiment, and thus they will not be described here again.

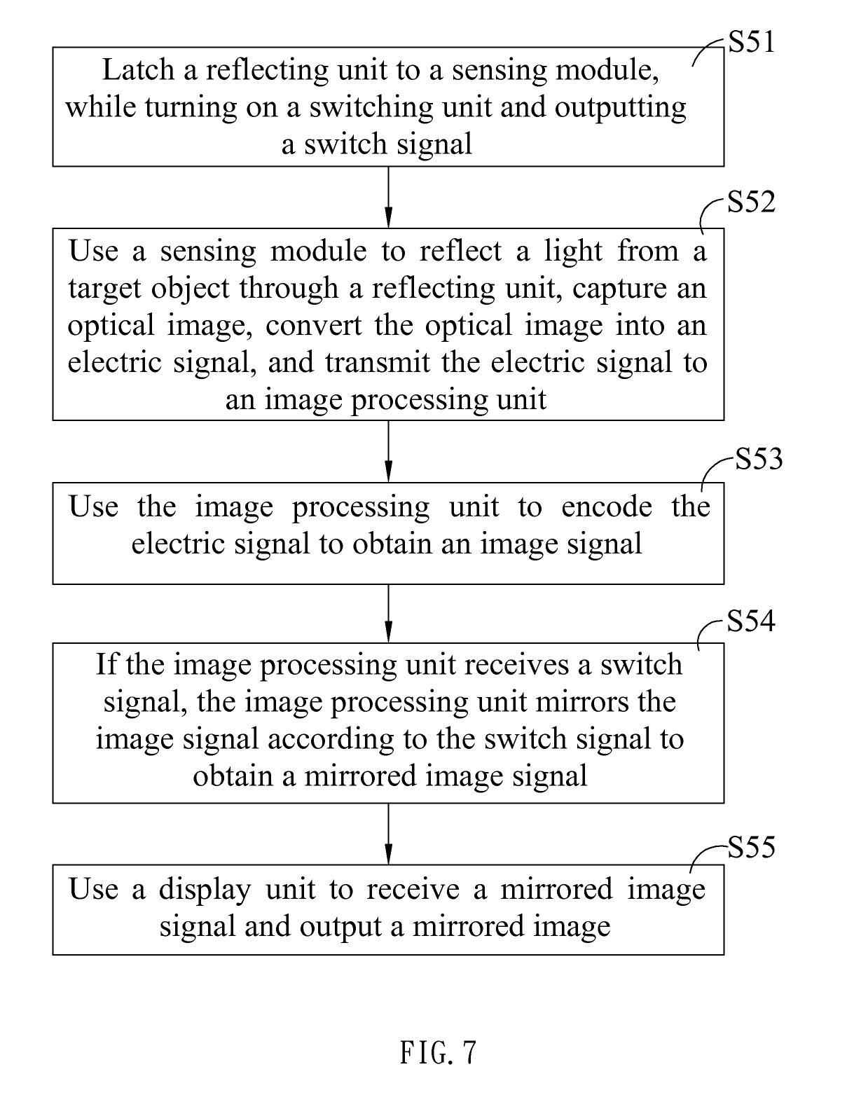

With reference to FIG. 7 for a flow chart of an image processing method applied for an endoscopic device in accordance with a second preferred embodiment of the present invention, the image processing method applied for the endoscopic device image comprises the following steps:

S51: Latch a reflecting unit to a sensing module, while turning on a switching unit and outputting a switch signal.

S52: Use a sensing module to reflect a light from a target object through a reflecting unit, capture an optical image, convert the optical image into an electric signal, and transmit the electric signal to an image processing unit.

S53: Use the image processing unit to encode the electric signal to obtain an image signal.

S54: If the image processing unit receives a switch signal, the image processing unit mirrors the image signal according to the switch signal to obtain a mirrored image signal.

S55: Use a display unit to receive a mirrored image signal and output a mirrored image.

In the image processing method applied for an endoscopic device in accordance with the second preferred embodiment of the present invention, the following method is used to achieve the effect of displaying the image in the same direction as the target object. Firstly, the reflecting unit is latched to the sensing module, and the switching unit is installed on a side of the sensing module, such that when the reflecting unit is latched, the switching unit is triggered. If the light source projects lights onto the target object, the light will be reflected to the camera lens through a reflecting unit to form an optical image. If the reflecting unit is not latched to the sensing module, the switching unit will not be triggered, and no switch signal will be outputted, so that the image signal will not be mirrored, but the display unit will receive an image signal and output an image. The installation and operation of the remaining components are the same as those of the first preferred embodiment, and thus will not be described here again.

While the invention has been described by device of specific embodiments, numerous modifications and variations could be made thereto by those generally skilled in the art without departing from the scope and spirit of the invention set forth in the claims.

In summation of the description above, the present invention can improve over the prior art and comply with the patent application requirements, and thus is duly filed for patent application.

What is claimed is:

1. An endoscopic device, comprising:
a casing;
a sensing module, for capturing an optical image according to a reflecting light reflected from a target object by a reflecting unit, and converting the optical image into an electric signal;
a switching unit, installed on a side of the casing;
an image processing unit, installed in the casing and electrically coupled to the switching unit, for encoding the electric signal into an image signal; and
a display unit, installed on another side of the casing;
when the switching unit is triggered, the switching unit outputs a switch signal, and the image processing unit performs a mirroring processing to the image signal to obtain a mirrored image signal according to the switch signal, and the display unit receives the mirrored image signal and outputs a mirrored image; and
when the switching unit is not triggered, the display unit receives the image signal and outputs an image.

2. The endoscopic device of claim 1, wherein the sensing module comprises a light source, a camera lens and an optical sensing element, and the light source projects lights to the target object, and the reflecting light is reflected from the target object to the camera lens by the reflecting unit to form the optical image, and the optical sensing element converts the optical image into the electric signal.

3. The endoscopic device of claim 1, wherein the mirrored image is an image formed by horizontally or vertically turning over the optical image.

4. The endoscopic device of claim 1, wherein the reflecting unit is installed on a side of the sensing module, and the reflecting unit is a reflective lens.

5. An image processing method applied for an endoscopic device, comprising the steps of:
using a sensing module to capture an optical image according to a reflecting light reflected from a target object by a reflecting unit, and converting the optical image into an electric signal, and transmitting the electric signal to an image processing unit;
using the image processing unit to encode the electric signal to obtain an image signal;
using the image processing unit to determine whether or not to output a switch signal by a switching unit;
mirroring the image signal according to the switch signal by the image processing unit to obtain a mirrored image signal when the image processing unit receives the switch signal; and
using a display unit to receive the mirrored image signal and output a mirrored image.

6. The image processing method applied for an endoscopic device as recited in claim 5, wherein the display unit receives the image signal and outputs an image if the image processing unit does not receive the switch signal.

7. The image processing method applied for an endoscopic device as recited in claim 5, wherein the sensing module comprises a light source, a camera lens and an optical sensing element, and the light source projects lights to the target object, the reflecting light is reflected from the target object to the camera lens by the reflecting unit to form the optical image, and the optical sensing element converts the optical image into the electric signal.

8. The image processing method applied for an endoscopic device as recited in claim 5, wherein the mirrored image is an image formed by horizontally or vertically turning over the optical image.

9. The image processing method applied for an endoscopic device as recited in claim 5, wherein the reflecting unit is installed on a side of the sensing module, and the reflecting unit is a reflective lens.

10. An endoscopic device, comprising:
a casing;
a sensing module, latched to a reflecting unit, for capturing an optical image according to a reflecting light reflected from a target object by a reflecting unit, and converting the optical image into an electric signal;
a switching unit, installed on a side of the sensing module;
an image processing unit, installed in the casing, and electrically coupled to the switching unit, for encoding the electric signal into an image signal; and
a display unit, installed on another side of the casing;
when the reflecting unit is latched to the sensing module, the reflecting unit triggers the switching unit, and the switching unit outputs a switch signal, and the image processing unit mirrors the image signal according to the switch signal to obtain a mirrored image signal, and the display unit receives the mirrored image signal and outputs a mirrored image; and
when the reflecting unit is separated from the sensing module, the reflecting unit does not trigger the switching unit, and the display unit receives the image signal and outputs an image.

11. The endoscopic device of claim 10, wherein the sensing module comprises a light source, a camera lens and an optical sensing element, and the light source projects lights to the target object, and the reflecting light is reflected from the target object to the camera lens by the reflecting unit to form the optical image, and the optical sensing element converts the optical image into the electric signal.

12. The endoscopic device of claim 10, wherein the mirrored image is an image formed by horizontally or vertically turning over the optical image.

13. The endoscopic device of claim 10, wherein the reflecting unit is installed on a side of the sensing module, and the reflecting unit is a reflective lens.

14. An image processing method applied for an endoscopic device, comprising the steps of:
using a reflecting unit to latch to a sensing module, while triggering a switching unit and outputting a switch signal;
capturing an optical image by the sensing module according to a reflecting light reflected by the reflecting unit from a target object, and converting the optical image into an electric signal by the sensing module, and transmitting the electric signal to an image processing unit;
using the image processing unit to encode the electric signal to obtain an image signal;
when the image processing unit receives the switch signal, mirrors the image signal according to the switch signal to obtain a mirrored image signal by the image processing unit; and
using a display unit to receive the mirrored image signal and output a mirrored image.

15. The image processing method applied for an endoscopic device as recited in claim 14, wherein if the reflecting unit is not latched to the sensing module, the switching unit will not be triggered.

16. The image processing method applied for an endoscopic device as recited in claim 14, wherein the sensing module comprises a light source, a camera lens and an optical sensing element, and the light source projects lights to the target object, and the reflecting light is reflected from the target object to the camera lens by the reflecting unit to form the optical image, and the optical sensing element converts the optical image into the electric signal.

17. The image processing method applied for an endoscopic device as recited in claim 14, wherein the electric signal is transmitted from the sensing module to the image processing unit through a transmission line.

18. The image processing method applied for an endoscopic device as recited in claim 14, wherein the mirrored image is an image formed by horizontally turning over the optical image.

19. The image processing method applied for an endoscopic device as recited in claim 14, wherein the mirrored image is an image formed by vertically turning over the optical image.

20. The image processing method applied for an endoscopic device as recited in claim 14, wherein the reflecting unit is installed on a side of the sensing module, and the reflecting unit is a reflective lens.
